(19) United States
(12) Patent Application Publication
(10) Pub. No.: US 2006/0241203 A1
(43) Pub. Date: Oct. 26, 2006

(54) Dental materials containing dendritic compounds

(75) Inventors: Klaus Ruppert, Maintal (DE); Andreas Grundler, Wuppertal (DE); Kurt Reischl, Merenberg (DE)

Correspondence Address: Norris, McLaughlin & Marcus P.A., 18th Floor, 875 Third Avenue, New York, NY, 10022, US

(73) Assignee: Heraeus Kulzer GmbH, Hanau (DE)

(21) Appl. No.: 11/398,382

(22) Filed: Apr. 5, 2006

(30) Foreign Application Priority Data
Apr. 20, 2005 (DE) 10 2005 018 451.0

(51) Int. Cl. A61K 6/10

(52) U.S. Cl. 523/109

(57) ABSTRACT

Dental materials containing at least one dendritic compound composed of a core and two shells, the core and first and second shells of which are joined to one another by means of polyurethane groups, and the second shell of which is modified by reaction with a (meth)acrylate, exhibit improved mechanical properties, in particular elasticity, flexural strength, and improved rheological properties before curing.

The invention relates to dental materials containing dendritic compounds.

### BACKGROUND OF THE INVENTION

Dendrimers or hyperbranched polymers are oligomeric or polymeric compounds which are built around a central molecule in a shell-like manner to produce a highly-branched, more or less symmetrical base structure, and which may be provided with functional groups on the outermost shell. Such compounds are referred to below as dendritic compounds.

The difference between dendrimers and hyperbranched polymers lies in their molecular symmetry: whereas dendrimers have a highly symmetrical structure as the result of strict reaction conditions, hyperbranched polymers sometimes exhibit asymmetry on account of static branching, but are much more economical to produce.

Dendrimers or hyperbranched polymers are used in particular for improving the mechanical properties of polymers. DE 44 43 702 A1 describes dendrimers in filler-containing, fine-grained, packable, i.e., amalgam-like, dental materials. US 200300114553 A1 relates to a similar curable and self-supporting dental material which may contain crystalline components having dendrimeric or hyperbranched structures. The use of dendrimers in rheopexic dental materials has been described in US 20020068771A1, for example.

### SUMMARY OF THE INVENTION

According to the present invention, specific dendritic compounds as described in Claims 1 through 4 are added to dental materials, resulting in dental materials according to Claims 1 through 4 which have surprisingly beneficial properties.

The invention consequently relates to dental materials containing at least one dendritic compound composed of a core and two shells,
the core and first and second shells of which are joined to one another by means of polyurethane groups,
and the second shell of which is modified by reaction with a (meth)acrylate.

### DETAILED DESCRIPTION

The polyurethane framework (core, first and second shell) is preferably formed by the known reaction of diisocyanates with trihydric or polyhydric alcohols. The diisocyanates preferably contain 4-12, particularly preferably 6-10, C atoms. Hexamethylene diisocyanate is very particularly preferred. Pentaerythritol, dipentaerythritol, or preferably trimethylolpropane may be considered as alcohols.

For modification of the outer shell, suitable (meth)acrylates are primarily meth)acrylates and acrylates containing hydroxy groups, preferably hydroxyethyl methacrylate.

Compounds represented by formula I have proven to be particularly suitable:

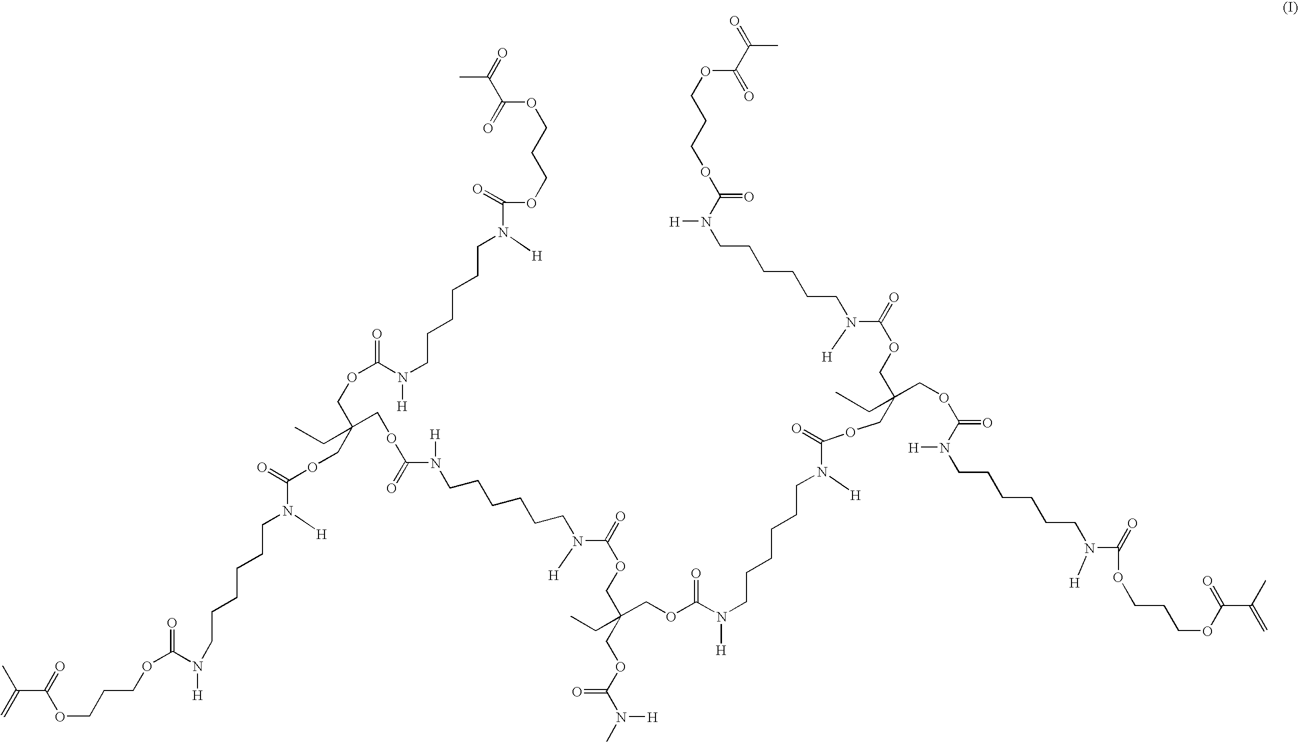

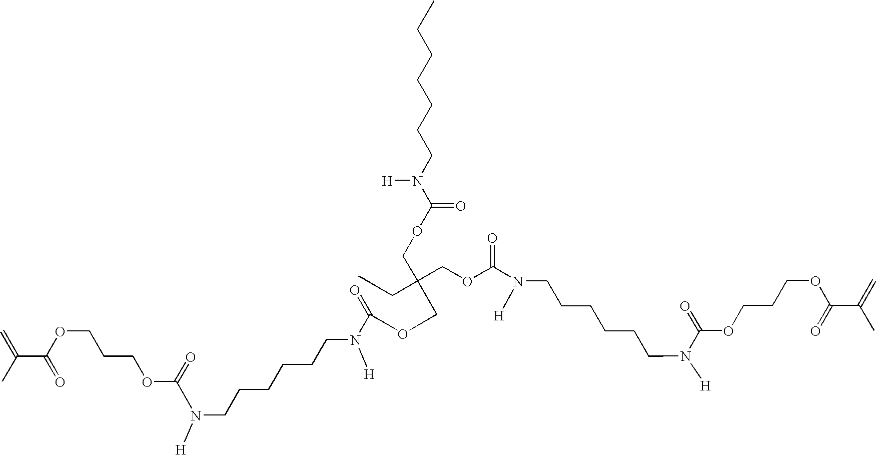

Characterizing features of compound I are as follows:
The compound is composed of two shells and one core.
The outer, second shell is modified 6 times with HEMA¹.
   ¹Hydroxyethyl methacrylate
The core and the shells are linked by means of polyurethane groups (obtainable from hexamethylene diisocyanate and trimethylolpropane).

Since compound I is the product of an oligomerization, this product does not always completely correspond to the ideal formula above. As a rule, the product is also obtained from other, less symmetrical compounds, which as hyperbranched oligomers are likewise dendritic compounds. In the present discussion, the terms “compound I” and of formula I encompass such less symmetrical compounds.

The dendritic compounds such as the compound of formula 1, for example, may be prepared according to methods known as such, for example, analogously to those described in J. Serb. Chem. Soc. 69, 441-453 (2004).

Dental materials which may be considered are known as such, and are described, for example, in Ullmann's Encyclopedia of Industrial Chemistry (CD-ROM 2002), Robert G. Craig, Dieter Welker, Dental Materials, Version 15, June 2000, in particular under “Cements” and “Synthetic Resins.” Composite materials, in particular filling composites, are preferred.

The dendritic compounds described above are used in particular for optimizing the mechanical properties, especially as elastifiers and/or rheology-improving agents. The flexural strength is increased by more than 10 MPa by, for example, compound I described below, compared to the same dental composite without the addition of compound I. In addition, undesirable polymerization shrinkage may be favorably influenced.

The dendritic compounds used according to the invention also improve the Theological properties of the dental materials before curing: under shear stress, i.e., as modeled, the viscosity of the paste decreases. As soon as the shearing action is discontinued, a significant increase in viscosity is observed (analogous to aerosils, but without their negative effect on product properties: aerosils specifically impair the transparency, and must be disaggregated).

A concentration of the dendritic compounds in the dental composite in the range of 1-10% by weight, preferably 1-5% by weight, very particularly preferably 1-3% by weight is advantageous.

### EXAMPLE

A monomer dispersion comprising the following components

| | |
|---|---|
| Bis-GMA | 14% by weight |
| TEDMA | 6% by weight |
| Non-agglomerated SiO₂ | 9% by weight |
| particles (20 nm) | |

is combined with 0.01% by weight BHT stabilizer (4-methyl-2,6-di-tert-butylphenol, CAS 128-37-0) and the initiators camphorquinone (0.3% by weight) and 0.2% by weight 2-ethylhexyl-4-dimethylaminobenzoate (Quantacure™ EHA, CAS 21245-02-3, Great Lakes Chemicals).

1% by weight of compound I was added, with moderate heating to 40° C. A silanized dental glass (Ba-Al silicate glass; 70.0% by weight) was then added. After thorough mixing, a dental material suitable as a filling composite was obtained.

1. Dental material comprising at least one dendritic compound, said dendritic compound being composed of a core and two shells,
the core and first and second shells of which are joined to one another by means of polyurethane groups,
and the second shell of which is modified by reaction with a (meth)acrylate.

2. Dental material according to claim 1, wherein the first and second shells are composed of
a polyurethane framework obtained from the reaction of hexamethylene diisocyanate and trimethylolpropane.

3. Dental material according to claim 1, wherein
said (meth)acrylate is hydroxyethyl methacrylate.

4. Dental material according to claim 1, wherein the dendritic compound essentially corresponds to formula I.

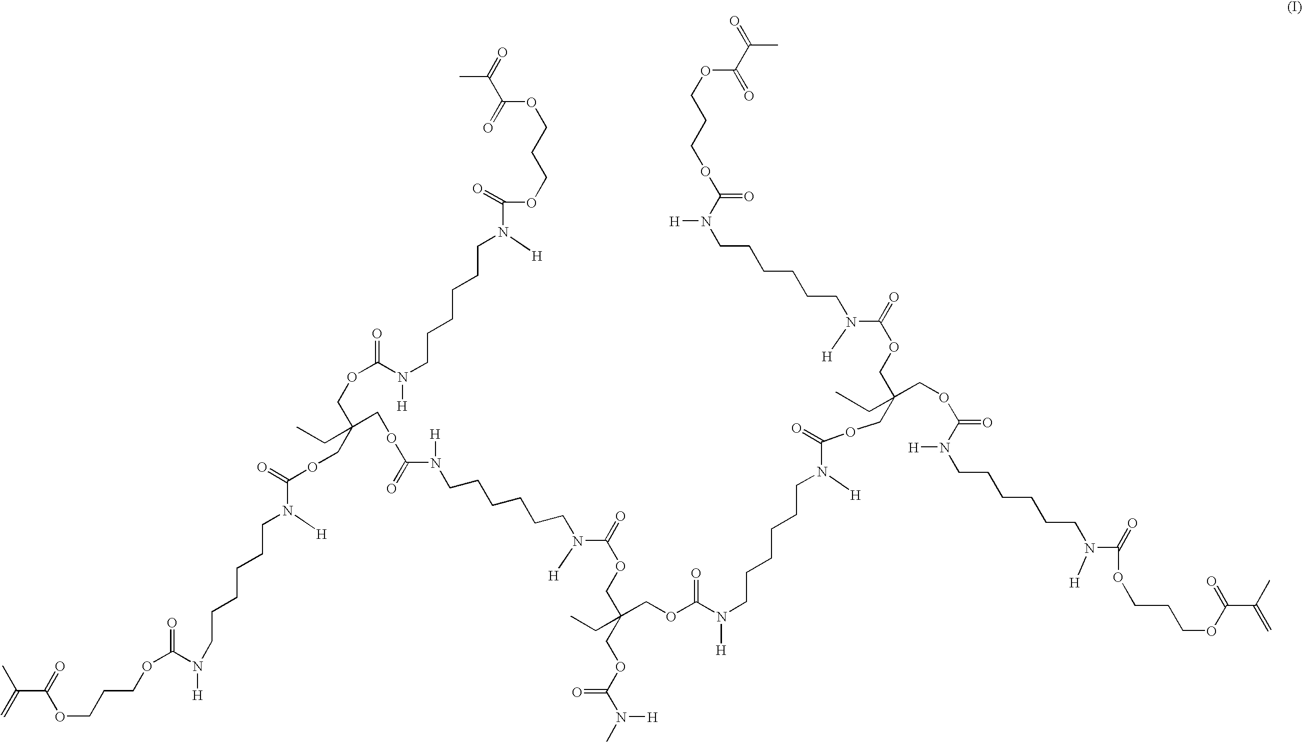

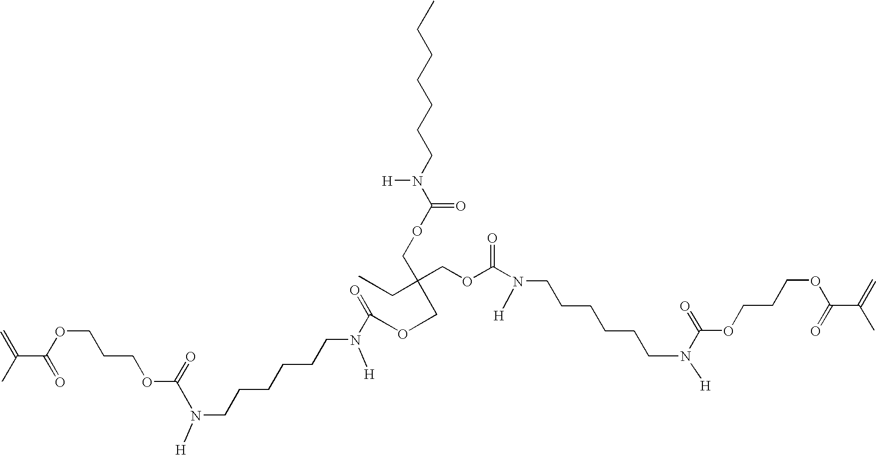

5. An elastifier for dental materials comprising the dendritic compound of claim 1.

6. A method for improving the rheological properties of dental materials which comprises adding the dendritic compound of claim 1 to said dental materials.
